(19) United States
(12) Patent Application Publication
(10) Pub. No.: US 2014/0205618 A1
(43) Pub. Date: Jul. 24, 2014

(54) HUMAN FERTILITY ENHANCEMENT WITH CORTISOL REDUCTION FOOD

(71) Applicants: John Edgar Menear, Santa Cruz, CA (US); Joseph Ramaekers, Aptos, CA (US)

(72) Inventors: John Edgar Menear, Santa Cruz, CA (US); Joseph Ramaekers, Aptos, CA (US)

(73) Assignee: CortControl, Inc., Santa Cruz, CA (US)

(21) Appl. No.: 13/999,875

(22) Filed: Mar. 29, 2014

Related U.S. Application Data
(63) Continuation-in-part of application No. 13/729,923, filed on Dec. 28, 2012.

(51) Int. Cl. A61K 38/17; A61K 31/716; A23L 1/29; A61K 35/74

(52) U.S. Cl. CPC A61K 38/1709; A61K 35/744; A61K 31/716; A23L 1/296
USPC 424/184.1

(57) ABSTRACT

A method of using a medical food to increase human fertility. This medical food consists of transfer factor, lactic acid generating bacteria, and glucans in appropriate combinations. The medical food, administered correctly, reduces cortisol levels. Progesterone increases as cortisol decreases, and progesterone is needed to support pregnancy and ovulation. Dosage amounts are adjusted for client weight. Typically, consumption of the medical food begins several days before planned conception.

### CROSS-REFERENCE TO RELATED APPLICATIONS

This application is a continuation-in-part of U.S. application Ser. No. 13/729,923 filed Dec. 28, 2012. Priority is also claimed to U.S. Provisional application 61/964,100 filed Dec. 24, 2013.

### FIELD OF THE INVENTION

The invention relates to a method of administering food compositions and formulations comprising at least transfer factor and lactic acid generating bacteria to increase human fertility. In particular, fertility is increased by reducing cortisol in men and women with the administered formulations.

### BACKGROUND OF THE INVENTION

In the United States, the number of women ages 15-44 with impaired fecundity (impaired ability to get pregnant or carry a baby to term) is 6.7 million. Multiple fertility clinics exist to help women with fertility issues.

Current research shows repeatedly that stress is an important factor. One fertility clinic broadcasts the following message: “The best fertility treatment is beating stress”. A 2009 study at Emory University concluded that “We think there are women who have sub-clinical forms of stress and who are infertile as a result . . . ”

Stress is a broad term and carries connotations. Different observers may define stress in different symptomatic ways. Fortunately, stress is associated with a chemical marker. Cortisol is the stress hormone, and cortisol measurement allows stress to be quantified.

The veterinary field was first to realize the importance of correlating high cortisol to infertility. Low calving rates in cattle herds represent a large loss to the rancher. Birth rates increased when cortisol levels dropped.

The experience of livestock is significant because the hormonal mechanism of stress is the same in all mammals. Humans generate the stress hormone, cortisol, in the same way as a goat, pig, cow, horse, or monkey. Basically, the pituitary releases ACTH (adrenocorticotropic hormone). Then ACTH stimulates the adrenal cortex to secrete cortisol.

Cortisol has a positive value in the short term. It energizes the body for a fight or flight situation. But a cortisol excess over a long time is destructive. Fertility is one of the casualties.

One mechanism of cortisol-induced infertility is referred to as the “progesterone steal”. In this scenario, the body's production of excess cortisol uses progesterone as a reactant. The end result is a low level of progesterone, which is needed to support the pregnancy. A second mechanism of cortisol-induced infertility is referred to as “polycystic ovary syndrome”, which interferes with egg production. Again, high cortisol and low progesterone are involved.

However, this application is not limited by any mechanism that relates cortisol to infertility. Inferences in this instant application are more supported by test data than by theories.

Human information can be extracted from mammal studies. All mammals have a cortisol producing glandular system that mimics humans. Basically, the pituitary gland secretes ACTH (adrenocorticotropic hormone), which then stimulates the adrenal glands to release cortisol. The success with mammals is transferrable to humans.

Cattle studies have repeatedly demonstrated that feeding a mixture of transfer factor and lactic acid generating bacteria increases fertility.

Fertility clinics address infertility with a variety of treatments. Some of these treatments may have side effects. In contrast, the mixture of transfer factor and lactic acid generating bacteria is a food, not a drug. Foods take longer to confer benefits than drugs, but foods comprising transfer factor and lactic acid generating bacteria are inherently safe.

The consuming public now understands that foods possess more than basic nutrition (protein, carbohydrate, fat, etc.). For example, 95% of consumers agree that “certain foods have health benefits that go beyond basic nutrition and may reduce the risk of disease or other health concerns”. More than 50% of consumers believe that foods can replace the use of drugs.

The Federal Drug Administration acknowledges this trend with the relatively new category of “Medical Foods”. Medical Foods should be administered and monitored by a doctor, nutritionist, nurse, medical technician or equivalent health care professional.

A food-based fertility treatment option is needed that addresses root causes, and avoids side effects. A food based treatment may be used in conjunction with other treatments.

### BRIEF SUMMARY OF THE INVENTION

This instant invention is a method of treating infertility with foods that reduce human cortisol levels. Reduced cortisol levels correlate with higher progesterone levels and enhanced fertility. Progesterone means “for gestation”. Progesterone is necessary for healthy egg production, and the egg's stable attachment to the uterus. Progesterone is further necessary to feed and nourish the uterus during pregnancy, and, hence, support the fetus. Low progesterone is associated with early miscarriage and failure to reach full term.

Following is a condensed summary of the invention. By necessity, details are omitted in order to simply state the essence of the invention. Omitted details within this section should not be construed in a way that limits the scope of the invention.

One preferred food composition used to treat infertility is a mixture of transfer factor and lactic acid generating bacteria. This composition is described in U.S. Pat. No. 6,962,718 issued to Joseph Ramaekers.

Another preferred food composition used to treat infertility is a mixture of transfer factor, lactic acid generating bacteria, and glucans. Glucans may be present as mushrooms. This composition is encompassed by domineering U.S. Pat. No. 6,962,718, and contains one additional limitation (glucans).

Either preferred composition may be augmented with additional additives.

Dosages of medical foods may be adjusted for patient weight.

The relative proportion of transfer factor, lactic acid generating bacteria, and glucans within a dose may vary. Although typical proportions can be recited, proportions may be modified to best serve each individual.

For some clients, transfer factor, lactic acid generating bacteria, and glucans are taken together. For other clients, transfer factor, lactic acid generating bacteria, and glucans are taken at different times during the day or week. Component separation and consumption at different times are within the scope of this invention.

Typical method-of-use steps include some or all of the following: (1) deciding that high cortisol levels may be an infertility issue, (2) selecting the medical food dosage level, (3) beginning consumption of the medical food before planned conception, (4) continuing the medical food 10 days to nine months into the pregnancy, and (5) measuring cortisol levels between conception and birth.

Patent application Ser. No. 13/729,923 solved the infertility issue for livestock with (1) a combination of transfer factor and lactic acid generating bacteria, or (2) a combination of transfer factor, lactic acid generating bacteria, and glucans. Ramaekers (in Ser. No. 13/729,923) determined that feeding these combinations to livestock increased the calf birthrate, and lowered cortisol levels. For testing, bovine populations of 200-300 were divided into randomized test and control groups.

This instant application extends the fertility success of patent application Ser. No. 13/729,923 to human fertility.

Transfer of fertility data from livestock to humans is based on three facts. First, cortisol biochemistry is the same among mammal species. Second, transfer factor functions the same across species. Third, lactic acid generating bacteria and glucans are utilized nutritionally the same way.

Informal studies also demonstrate the connection between lowered cortisol and either (1) a combination of transfer factor and lactic acid generating bacteria, or (2) a combination of transfer factor, lactic acid generating bacteria, and glucans. Feeding these foods to stressed athletes lowers the symptoms of cortisol excess. College students consume these foods before final examinations to lower cortisol and get a better night's sleep.

The connection in humans and other mammals is clear from a preponderance of data. Lowered cortisol measurements correlate to consuming transfer factor, lactic acid generating bacteria, and/or glucans. In turn, fertility is enhanced by lowering cortisol. Other stress hormones may also be lowered by consuming transfer factor, lactic acid generating bacteria, and/or glucans.

### BRIEF DESCRIPTION OF THE DRAWINGS

FIG. 1 is a diagram of cortisol generation in mammals.

FIG. 2 is a diagram of how cortisol production consumes progesterone, which decreases the probability of a successful pregnancy.

FIG. 3 graphically shows that a combination of transfer factor, lactic acid generating bacteria, and glucans reduces cortisol production in mammals. Evening cortisol is presented.

FIG. 4 graphically shows that a combination of transfer factor, lactic acid generating bacteria, and glucans reduces cortisol production in mammals. Morning cortisol is presented.

### DETAILED DESCRIPTION OF THE INVENTION

Transfer factor is produced by leucocytes and lymphocytes. Transfer factor comprises small water soluble polypeptides of about 44 amino acids that stimulate or transfer cell mediated immunity from one individual to another.

The properties, characteristics and processes for obtaining transfer factor or transfer factors are discussed in U.S. Pat. Nos. 4,816,563; 5,080,895; 5,840,700, 5,883,224 and 6,468,534, the contents of which are hereby incorporated by reference into the present application.

Alternative sources of transfer factor include avian transfer factor, ova transfer factor, and colostrum from goats, pigs, horses and humans. This listing is not complete. In addition, combinations of transfer factors from any number of sources may be used in fertility formulations.

In certain embodiments of fertility enhancement, substantially purified transfer factor has a molecular weight of less than 10,000 Daltons.

Transfer factor is commercially available, and known to be safe.

Lactic acid generating bacteria is an important component of the infertility medical food, and is GRAS (generally recognized as safe). Lactic acid generating bacteria support digestion and brain health. Lactic acid generating bacteria provide healthful effects that are found in non-pasteurized sauer kraut and cod liver oil. Within the intestinal tract, lactic acid generating bacteria are beneficial. It has been estimated that 80% of human health depends on beneficial intestinal bacteria.

A human body becomes stressed by poor digestion, and cortisol levels will reflect that stress. Lactic acid generating bacteria helps reduce cortisol via improved digestion.

Glucans (polysaccharides) are known to support the immune system. When combined with transfer factor and lactic acid generating bacteria, a synergy is created. The combined effect on fertility is greater than the effect predicted from summing the individual components.

Although much emphasis is placed on female clients, male reproductive health and function is also improved by the feeding formulations comprising (1) transfer factor and lactic acid generating bacteria or (2) transfer factor, lactic acid generating bacteria, and glucans. This improved function may include quantity or quality of sperm produced by the male. One-third to one-half of infertility originates with the male.

Once diagnosed, the infertility treatment should address the underlying cause.

Most likely medicines will be prescribed, but medicines can have undesirable side effects.

In some cases, progesterone supplementation may be tried. But research has shown that this is not the ideal solution. Progesterone levels do not return to normal without first addressing cortisol levels.

One preferred food composition to-treat infertility is a mixture of transfer factor and lactic acid generating bacteria. This composition is patented (Joe Ramaekers, U.S. Pat. No. 6,962,718, claim 6, issued Nov. 8, 2005). The method of using transfer factor and lactic acid generating bacteria for treating infertility can be viewed as an improvement limitation on the existing commonly-owned composition patent. The improvement comprises the method of using the patented composition. An improvement claim is presented in the claims section.

Another preferred food composition used to treat infertility is a mixture of transfer factor, lactic acid generating bacteria, and glucans. Glucans may be present as mushrooms. The method of using transfer factor, lactic acid generating bacteria, and glucans for treating infertility can also be viewed as two improvements to U.S. Pat. No. 6,962,718. The two improvements (limitations) are (1) the addition of glucans, and (2) the method of using the patented composition. An improvement claim is presented in the claims section.

Either preferred composition may be augmented with additional additives. Example additives are minerals, probiotics, prebiotics, dimethyl glycine, ascorbic acid, Vitamin A, Vitamin D3, Vitamin E, Vitamin B1, Vitamin B2, Vitamin B12, dipotassium phosphate, potassium chloride, magnesium sulfate, calcium pantothenate, minerals, antioxidants, amino acids, nutraceuticals, inositol hexaphosphate (Ip6), mannans, olive leaf extract, and phytosterols. In certain preferred embodiments, mannans are derived from Aloe vera. In certain preferred embodiments, phytosterols may be derived from soya bean.

Probiotics additives include, but are not limited to B. subtlis, B. longum, B. thermophilium, B. coagulans, E. faecium, and S. cerevisia, L. casei, L. plantarum, Pediococccus acidilacticii, Kluyveromyces marxianus fragillis and combinations thereof.

The above listings do not include all possible additives. The food compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

The relative proportion of transfer factor, lactic acid generating bacteria, and glucans within the composition may vary widely.

However, some reasonable weight ranges for transfer factor are 0.05-50 mg/pound of body weight. Reasonable weight ranges for lactic acid generating bacteria are 0.47-10 mg/pound of body weight. This is based on a nominal live count of 2.5×10⁶ CFU/Ounce. Reasonable weight ranges for glucans are 0.1-10 mg/pound of body weight.

The method of using the fertility enhancement foods may have some or all of the following steps:
(1) determine with measurement that cortisol is a contributing factor to infertility,
(2) select the correct proportion of transfer factor, lactic acid generating bacteria, and glucans,
(3) choose the correct dosage level,
(4) select a feeding frequency between five times per day and once per week,
(4) begin consumption before the next ovulation cycle,
(5) periodically measure serum or salivary cortisol levels and adjust food dosage, and
(6) continue consumption until a stable-pregnancy is achieved.

Some documented veterinary fertility improvements follow. They are presented to establish expectations where fertility foods are applied to human fertility utilization. There are large numbers of improved fertility cases documented in animals. Cortisol reduction is wholly or partially the reason for success. Other stress markers may also be lowered. Because the cortisol production method is the same for humans and other mammals, parallel fertility successes are available to humans.

### Example 1

A young bull nine months old was evaluated to have no live semen. The animal was then administered one ounce of the fertility formula daily for one month. After one month, 75 ampules of viable semen were collected from the animal. This is an above average yield.

### Example 2

Cattle breeding without administration of transfer factor formulation yielded about 75% conception. Addition of the transfer factor formulation increased the rate of conception to 98%. Observations were consistent with lowered stress.

### Example 3

Thirty-five (35) mature Hampshire ewes in Santa Rosa with fertility difficulty demonstrated conception at about 40% for several years. Administration of one ounce of the transfer factor formulation on Days 6 and 7 prior to breeding increased conception to about 95%.

### Example 4

A similar case was observed with donor cows. Without the fertility formula the best flush (donor) cow yielded 6 to 8 eggs; with usually only 1 or 2 eggs attaining Grade 1. With the fertility formula, the same cow yielded 12 eggs; 10 of these were Grade 1. Initial high cortisol was suspected due to stress.

### Example 5.

In a commercial beef operation 100 cows were administered one ounce of fertility formulation 6 to 7 days before breeding. Conception improved by 30% with these protocols.

### Example 6

Emory University has correlated both human and monkey infertility to cortisol levels. Studies are continuing.

The above animal fertility results are a very small fraction of the animal data available. A preponderance of test data supports the effect of administering transfer factor, lactic acid generating bacteria, and/or glucans to overcome infertility. Limited animal data is deemed sufficient because this instant application is focused on human fertility rather than animal fertility.

Most human studies focus on females with high cortisol. In hindsight, there is good reason for this. Miscarriages due to cortisol tend to occur in mammals within 3 weeks of conception. Any cortisol increases during the first few weeks after conception would have to be maternal because embryos cannot produce glucocorticoids during that period.

This is supported by a 2006 finding that suggests pregnancy may be particularly sensitive to maternal stress during the placentation period. The Proceedings of the National Academy of Science (Mar. 7, 2006) estimated the average time from ovulation to fetal loss in unsuccessful pregnancies was 16 days.

It is noteworthy that most pregnancy studies document only clinical pregnancies, which are defined as 6 weeks and longer. Unfortunately, data about early miscarriage is probably under-reported.

However, this does not negate the value of fertility foods for males with high cortisol. The highest probability of success derives from both male and female using the fertility food composition.

The interaction of cortisol and fertility is shown in four figures.

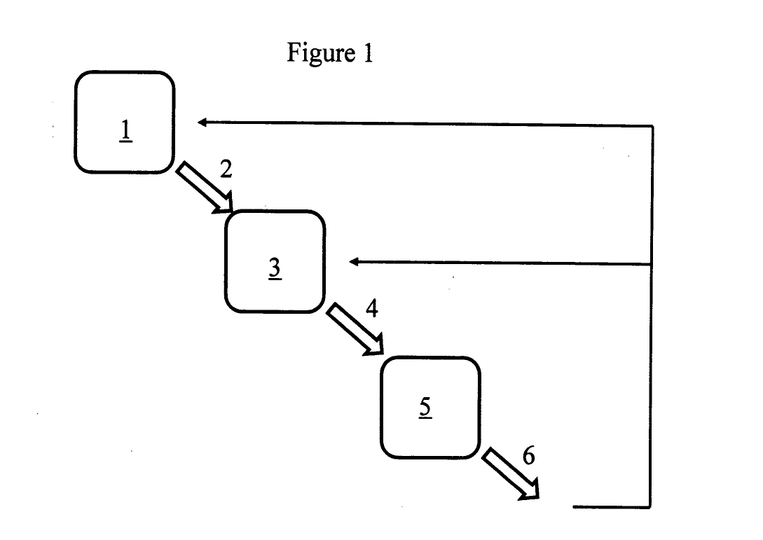

FIG. 1 shows the cortisol production sequence. FIG. 1 applies to humans and other mammals. The hypothalamus 1 releases corticotropin releasing hormone 2, which causes the anterior pituitary gland 3 to secrete adrenocorticotropic hormone 4. Adrenocorticotropic hormone 4 travels to the adrenal cortex 5, which responds by producing cortisol 6. Levels of cortisol are controlled by negative feedback loops 7.

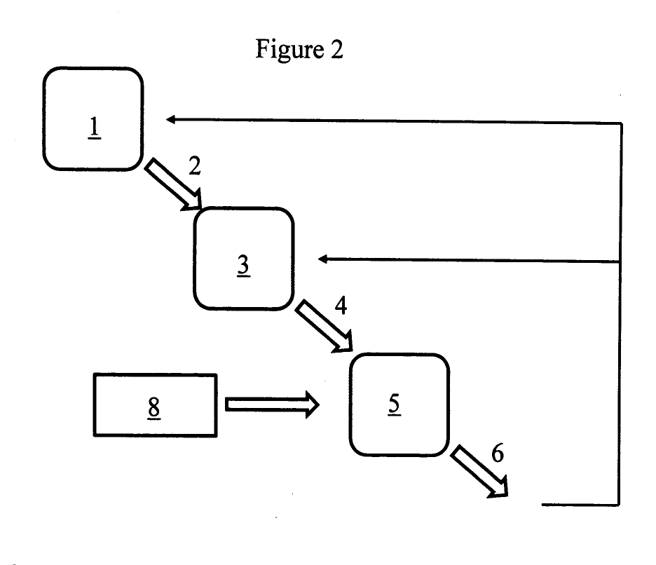

FIG. 2 diagrams how progesterone 8 is consumed as cortisol 6 is produced by the adrenal cortex 5. The result of over-production of cortisol is sub-normal progesterone levels. This is one known cause of infertility. However, this instant application is not limited by the “progesterone steal” mechanism. Successful intervention with transfer factor, lactic acid generating bacteria, and/or glucans is based on experimental evidence rather than theory.

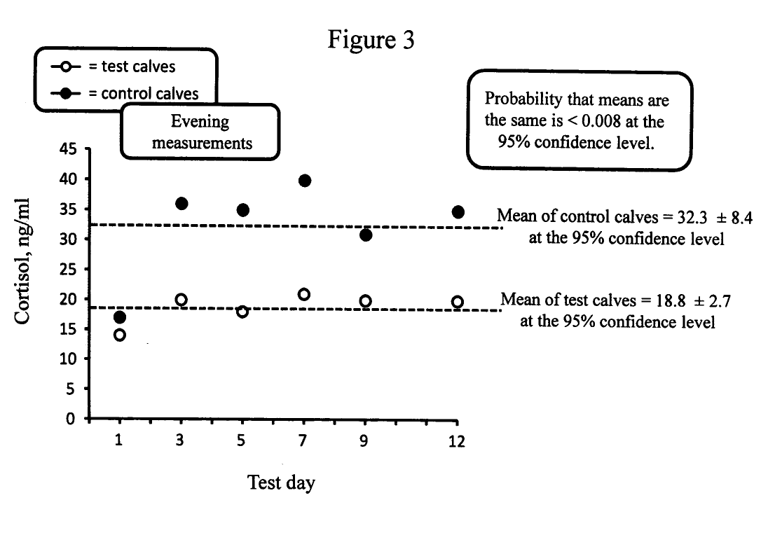

FIG. 3 graphically shows a drop in evening cortisol for stressed calves when fed a mixture of transfer factor, lactic acid generating bacteria, and/or glucans. This graph is provided to show how cortisol is decreased in mammals by feeding the appropriate mixture.

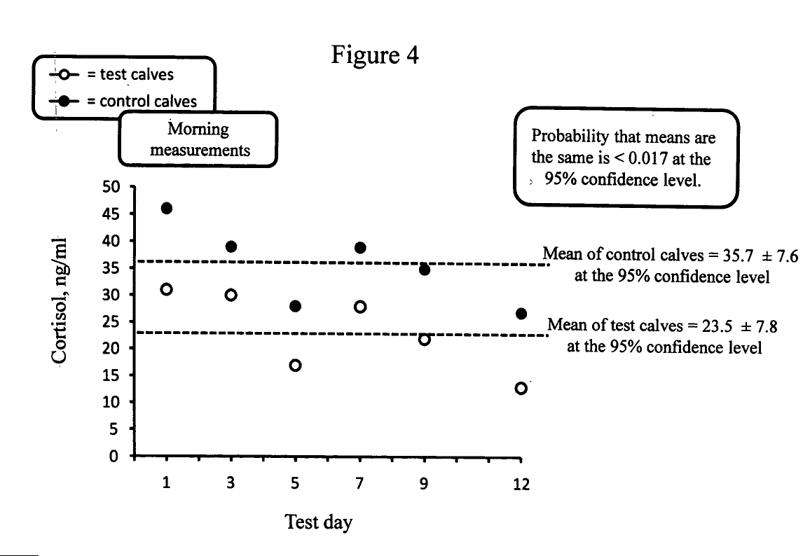

FIG. 4 graphically shows a drop in morning cortisol for stressed calves when fed a mixture of transfer factor, lactic acid generating bacteria, and/or glucans. As before, this graph is provided to show how cortisol is decreased in mammals by feeding the appropriate mixture.

Other stress hormones and chemical markers may also benefit from a mixture of transfer factor, lactic acid generating bacteria, and/or glucans. Examples include alpha amalyase and T4 measurements of thyroid function.

The invention claimed is:

1. A food-based method for improving fertility of a human when high cortisol levels are contributing to infertility comprising:
combining transfer factor transfer and lactic acid generating bacteria to create a food supplement, where
said transfer factor includes some polypeptides with a molecular weight below 10,000 Daltons;
feeding said food supplement to said human, where
a dosage level is chosen based on said human's weight, and
the frequency of said feeding is between five times per day and once per week; and
continuing said feeding until a stable pregnancy is achieved.

2. The food-based method of claim 1 wherein glucans are added to said food supplement.

3. The food-based method of claim 2 wherein said glucans are derived from natural or hybrid mushrooms.

4. The food-based method of claim 2 further comprising measuring cortisol levels periodically.

5. The food-based method of claim 4 further comprising adjusting said dosage levels based on said measuring.

6. The food-based method of claim 2 further comprising changing the relative proportions of transfer factor, lactic acid generating bacteria, and glucans included within said food supplement.

7. The food-based method of claim 2 wherein said transfer factor in each said dosage is present at 0.05 to 50 mg per pound of human body weight.

8. The food-based method of claim 2 wherein said lactic acid generating bacteria in each said dosage is present at 0.47 to 10 mg per pound of human body weight.

9. The food-based method of claim 8 wherein said lactic acid generating bacteria has a live count of 2.5 million colony forming units per ounce.

10. The food-based method of claim 2 wherein said glucans in each said dosage is present at 0.1 to 10 mg per pound of human body weight.

11. The food-based method of claim 1 wherein said continuing step lasts between 10 days and 9 months.

12. The food-based method of claim 2 wherein transfer factor transfer, lactic acid generating bacteria, and glucans are separated and consumed at different times within a one week period.

13. An improvement upon U.S. Pat. No. 6,962,718, claim 6 (issued Nov. 8, 2005 to Joseph Ramaekers, a current inventor) which recites—
A formulation comprising pharmaceutically acceptable transfer factor and a pharmaceutically acceptable lactic acid generating bacteria wherein the amount of said transfer factor is from 10 mg to 10,000 mg per ounce of formulation;
wherein the improvement comprises the following method-of-use limitation,
feeding said formulation to human infertility patients to increase fertility.

14. The improvement claim in claim 13, wherein said feeding includes at least one step selected from a group including:
(a) adjusting proportions of transfer factor transfer and said lactic acid generating bacteria within said formulation to create an infertility food supplement,
(b) choosing a dosage level of said formulation based on said infertility patient's weight,
(c) feeding said infertility patient a dosage between five times per day and one time per week,
(d) measuring said infertility patient's cortisol levels periodically,
(e) adjusting said dosage levels based on periodic cortisol measurements, and
(f) monitoring multiple stress hormones.

15. The improvement claim in claim 13, wherein glucans are added to known said formulation.

16. The improvement claim in claim 15, wherein said glucans are present between at 0.1 to 10 mg per pound of patient's body weight.

17. The improvement claim 13 wherein the patient is male.

18. The improvement claim 13 wherein the patient is female.

19. A food-based method for improving fertility of a human when high cortisol levels are contributing to infertility comprising:
combining transfer factor transfer and lactic acid generating bacteria to create a food supplement, where
said transfer factor includes some polypeptides with a molecular weight below 10,000 Daltons;
feeding said food supplement to said human, where
a dosage level is chosen based on said human's weight, and
the frequency of said feeding is between five times per day and once per week; and
utilizing a health care professional or doctor to supervise said feeding throughout the periods of conception and pregnancy.

20. A food-based method of claim 19 wherein said health care professional includes a doctor, a fertility clinic technician, a nurse, or a nutritionist.
